(19) United States
(12) Patent Application Publication
(10) Pub. No.: US 2025/0170096 A1
(43) Pub. Date: May 29, 2025

(54) OXPHOS INHIBITORS FOR USE IN TREATING CANCER

(71) Applicant: ImmunoMet Therapeutics, Inc., Houston, TX (US)

(72) Inventor: Dean WELSCH, Houston, TX (US)

(21) Appl. No.: 18/838,689

(22) PCT Filed: Feb. 16, 2023
(86) PCT No.: PCT/US2023/013180

§ 371 Date: Aug. 15, 2024

Related U.S. Application Data
(60) Provisional application No. 63/268,111, filed on Feb. 16, 2022.

(51) Int. Cl. A61K 31/40; A61K 31/337; A61K 31/7068; A61P 35/00; A61P 35/04

(52) U.S. Cl. CPC A61K 31/40; A61K 31/337; A61K 31/7068; A61P 35/00; A61P 35/04

(57) ABSTRACT

This invention features methods of treating a cancer in a subject, where the methods include administering an oxidative phosphorylation (OXPHOS) inhibitor to the subject, and where the cancer is refractory to a first line of treatment or is recurrent.

### PRIORITY CLAIM

This application claims benefit of the filing date of U.S. provisional application 63/268,111, filed Feb. 16, 2022, the content of which is hereby incorporated by reference in its entirety.

### FIELD OF THE INVENTION

In general, the present invention relates to methods of treating cancer in a subject in need thereof with an inhibitor of oxidative phosphorylation (OXPHOS). The invention further relates to treating refractory or recurring cancer in a subject in need thereof with an inhibitor of oxidative phosphorylation.

### BACKGROUND OF THE INVENTION

Cancer, a group of diseases caused by the harmful, abnormal, uncontrolled, and undesirable growth of cells, occurs in multiple tissues and organs, including the breasts, the lungs, the bladder, the colon, the rectum, the uterus, the testes, the kidneys, the blood, the lymphatic system, the liver, the bile ducts, the skin, the pancreas, the prostate, the thyroid gland, the brain, and the stomach. Globally, approximately 10,000,000 individuals die of cancer per year. Current therapies for treating cancer may fail to treat recurrent cancer. Accordingly, there is an ongoing need for improved therapies for treating cancer.

### SUMMARY OF THE INVENTION

The present invention provides methods of treating cancer by administering an oxidative phosphorylation (OXPHOS) inhibitor, where the cancer is refractory or is recurrent. The present invention also provides methods using the compound of Formula 1 below or a pharmaceutically acceptable salt thereof

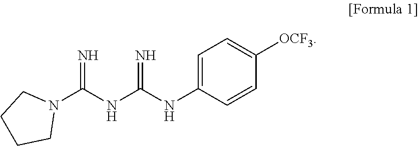 [SMILES: N=C(NC(=N)N1CCCC1)Nc1ccc(OC(F)(F)F)cc1]

for treating cancer.

In one aspect, the invention features a method of treating a cancer in a subject, where the method includes administering an oxidative phosphorylation (OXPHOS) inhibitor to the subject, and where the cancer is refractory to a first line of treatment or is recurrent.

In one embodiment, the first line of treatment is the standard of care for the cancer. In other embodiments, the cancer is a carcinoma, a sarcoma, a leukemia, or a lymphoma. In another embodiment, the cancer is a solid tumor. In an additional embodiment, the cancer is a metastatic cancer.

In some embodiments, the leukemia is an acute lymphocytic leukemia, a chronic lymphocytic leukemia, a myeloid leukemia, or a chronic myeloid leukemia. In some embodiments, the lymphoma is a Hodgkin lymphoma or a non-Hodgkin lymphoma. In other embodiments, the cancer is a breast cancer, lung cancer, non-small cell lung cancer, small cell lung cancer, bladder cancer, colon cancer, rectal cancer, uterine cancer, vaginal or vulvar cancer, testicular cancer, kidney cancer, hepatic cancer, bile duct cancer, cholangiocarcinoma, gallbladder cancer, melanoma, pancreatic cancer, pancreatic ductal adenocarcinoma, BRAF-mutant metastatic melanoma, prostate cancer, castrate-resistant prostate cancer, thyroid cancer, glioblastoma, endometrial cancer, ovarian cancer, cervical cancer, brain cancer, glioblastoma, liver cancer, colorectal cancer, skin cancer, basal cell carcinoma, squamous cell carcinoma, melanoma, gynecologic cancer, head or neck cancer, mesothelioma, myeloma, esophageal cancer, or gastric cancer.

In a particular embodiment, the OXPHOS inhibitor is a compound of Formula 1 or a pharmaceutically acceptable salt thereof.

In some embodiment, the compound of Formula 1 is formulated as a tablet or a capsule. In other embodiments, the tablet or capsule is formulated for delayed release. In further embodiments, the tablet or capsule includes an enteric coating. In some embodiments, the compound of Formula 1 is administered at a dose of about 100 mg, 200 mg, 400 mg, 800 mg, or 1200 mg per day. In a particular embodiment, the compound of Formula 1 is administered at a dose of about 800 mg per day.

In other embodiments, the OXPHOS inhibitor is administered in combination with a second therapeutic compound.

In another aspect, the invention features a method of treating pancreatic ductal adenocarcinoma (PDAC), where the method includes administering a pharmaceutical composition to a subject in need thereof with a therapeutically effective dose, and where the composition includes a compound of Formula 1 or a pharmaceutically acceptable salt thereof as an active ingredient.

In one embodiment, the compound of Formula 1 is administered in combination with Gemcitabine and Abraxane.

In an additional aspect, the invention features a method of treating BRAF-mutant melanoma, where the method includes administering a pharmaceutical composition to a subject in need thereof with a therapeutically effective dose, and where the composition includes a compound of Formula 1 or a pharmaceutically acceptable salt thereof as an active ingredient.

In some embodiments, the compound of Formula 1 is administered in combination with encorafenib and binimetinib. In other embodiments, the BRAF-mutant melanoma is metastatic. In some embodiments, the metastasis is to the central nervous system. In a particular embodiment, the metastasis is to the brain. In another embodiment of this aspect, the method includes use of radiation and/or administration of temozolomide.

In another aspect, the invention features a method of treating castrate-resistant prostate cancer (CRPC), where the method includes administering a pharmaceutical composition to a subject in need thereof with a therapeutically effective dose, and where the composition includes a compound of Formula 1 or a pharmaceutically acceptable salt thereof as an active ingredient.

In some embodiments, the subject did not respond to at least one line of immunotherapy. In other embodiments, the compound of Formula 1 is administered in combination with a checkpoint inhibitor. In a particular embodiment, the checkpoint inhibitor is a PD-1 inhibitor.

In some embodiments, the pharmaceutically acceptable salt is a salt of an acid selected from the group consisting of formic acid, acetic acid, propionic acid, lactic acid, butyric acid, isobutyric acid, trifluoroacetic acid, malic acid, maleic acid, malonic acid, fumaric acid, succinic acid, succinic acid monoamide, glutamic acid, tartaric acid, oxalic acid, citric acid, glycolic acid, glucuronic acid, ascorbic acid, benzoic acid, phthalic acid, salicylic acid, anthranilic acid, benzensulfonic acid, p-toluenesulfonic acid, methanesulfonic acid, dichloroacetic acid, aminooxy acetic acid, hydrochloric acid, hydrobromic acid, sulfuric acid, phosphoric acid, nitric acid, carbonic acid, and boric acid.

In a particular embodiment, the pharmaceutically acceptable salt is a salt of acetic acid.

In some embodiments, the subject is a human.

### Definitions

In this application, unless otherwise clear from context, (i) the term “a” may be understood to mean “at least one”; (ii) the terms “comprising” and “including” may be understood to encompass itemized components or steps whether presented by themselves or together with one or more additional components or steps; and (iii) the term “approximately” may be understood to permit standard variation as would be understood by those of ordinary skill in the art; and (iv) where ranges are provided, endpoints are included.

As used herein, the term “about” represents a value that is in the range of ±10% of the value that follows the term “about.” Reference to “about” a value or parameter herein includes (and describes) variations that are directed to that value or parameter per se. For example, a description referring to “about X” includes the description of “X”.

As used herein, the term “administration” refers to the administration of a composition (e.g., a compound or a preparation that includes an OXPHOS inhibitor) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, and vitreal.

An “effective amount” of a compound (e.g., an OXPHOS inhibitor) may vary according to factors such as the disease state, age, sex, and weight of the individual, and the ability of the compound to elicit the desired response. A therapeutically effective amount encompasses an amount in which any toxic or detrimental effects of the compound are outweighed by the therapeutically beneficial effects. An effective amount also encompasses an amount sufficient to confer benefit, e.g., clinical benefit.

As used herein, the term “subject” or “participant” or “patient” refers to any organism to which a compound or composition in accordance with the invention may be administered, e.g., for experimental, diagnostic, prophylactic, and/or therapeutic purposes. Typical subjects include any animal (e.g., mammals such as dogs, mice, rats, rabbits, pigs, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the subject; or enhancement or improvement of condition, disorder, or disease. Treatment includes eliciting a clinically significant response without excessive levels of side effects. Treatment also includes prolonging survival as compared to expected survival if not receiving treatment.

As used herein, the term “checkpoint inhibitor” means an inhibitor of a protein that determines whether an immune cell targets a cell for destruction, i.e., a checkpoint protein. Cancer cells express proteins on their surface that bind to bind to checkpoint proteins on T-cells. Binding between ligands on the surface of cancer cells and checkpoint inhibitors on immune cells causes the immune cell to fail to destroy the cancer cell. Checkpoint inhibitors block interactions between ligands on the surface of cancer cells and checkpoint inhibitors and allow the immune cell to destroy the ligand-bearing cancer cell.

As used herein, the term “PD-1 inhibitor” means an inhibitor of programmed death ligand-1 (PD1), which is a checkpoint inhibitor expressed on the surface of T cells and B cells. Exemplary and non-limiting PD-1 inhibitors include pembrolizumab, nivolumab, cemplimab, and dostarlimab.

As used herein, the term “recurrent cancer” means the growth of cancer cells that occurs within a subject after the subject has received treatment for the cancer. The cancer recurrence may be a local recurrence if the cancer reoccurs in the same place as it was originally found, a regional recurrence if the cancer reoccurs in the lymph nodes near the place the cancer was originally found, or a distant reoccurance if the cancer reoccurs in a different part of the body from where the cancer was originally found.

As used herein, the term “refractory cancer” is a cancer that does not respond to a first line of treatment, such as the standard of care, or that relapses after an initial response.

As used herein, the term “standard of care” means the treatment that a reasonably prudent physician would administer to a subject suffering from a disease or disorder.

As used herein, the term “carcinoma” refers to cancer that originates in the skin or in tissues that line or cover internal organs.

As used herein, the term “lymphoma” refers to cancer that originates in the cells of the immune system.

As used herein, the term “leukemia” refers to cancer that originates in blood cells or blood forming tissues.

As used herein, the term “sarcoma” refers to cancer that originates in bones or in the soft tissues of the body including cartilage, fat, muscle, blood vessels, fibrous tissue, connective tissue, or supportive tissue.

As used herein “enteric coating” is a polymer material or materials which encase the medicament core (e.g., one containing N1-pyrrolidine-N5-(3-trifluoromethoxy)phenyl biguanide, (the compound of Formula 1) or a pharmaceutically acceptable salt thereof) as they pass through the stomach. A suitable pH-sensitive polymer is one which will dissolve in the intestinal juices at the higher pH levels (pH greater than 4.5), such as within the small intestine and therefor permit release of the pharmacologically active substance in the regions of the small intestine and not in the upper portion of the GI tract, such as the stomach. Enteric coatings also protect the stomach from irritating pharmaceutical ingredients. Enteric coatings can be applied to a variety of formulations, including tablets, capsules, and microparticles. The enteric coating may be applied directly to the medicament core and may be an integral part of the tablet, capsule, or microparticle. Alternatively, the enteric coating may be separate from the medicament core where the coating, while encapsulating the medicament core, is not attached to the medicament core.

As used herein, “delayed release” means a pharmaceutical preparation, e.g., an orally administered formulation, which passes through the acidic environment of the stomach substantially intact and dissolves in the more basic environment of the small intestine. In some embodiments, delayed release of an active agent (e.g., N1-pyrrolidine-N5-(3-trifluoromethoxy)phenyl biguanide (the compound of Formula 1) or a pharmaceutically acceptable salt thereof) results from the use of a pH-sensitive enteric coating of an oral dosage form. An enteric coating can be combined with, for example, a delayed release formulation so as to extend the period of time over which drug is released.

As used herein, “effective amount” means the amount of an agent sufficient to effect beneficial or desired results in a patient, such as disease remission, and, as such, an “effective amount” depends upon the context in which it is being applied, including the age and weight of the patient, the nature of the disease, including the disease-affected organ(s), the disease status or level of activity, and other factors.

As used herein, “pharmaceutically acceptable salt,” represents those salts which are, within the scope of sound medical judgment, suitable for use in contact with the tissues of humans and animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds of the invention or separately by reacting the free base group with a suitable organic or inorganic acid. Representative acid addition salts include acetate, adipate, alginate, ascorbate, aspartate, benzenesulfonate, benzoate, bisulfate, bitartrate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. In one embodiment, the pharmaceutically acceptable salt is a salt of acetic acid.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the Drawings and Description below. Other features, objects, and advantages of the invention will be apparent from the Description and from the claims.

### BRIEF DESCRIPTION OF THE DRAWINGS

FIG. 1A is a plot showing pre- and post-treatment biopsies in 84 unique cancer therapies.

FIG. 1B is a plot showing an increase in OXPHOS in post-treatment biopsies relative to pre-treatment biopsies and no commensurate increase in glycolysis in post-treatment biopsies relative to pre-treatment biopsies.

FIG. 1C is a plot showing an increase in OXPHOS following leuprolide treatment in breast cancer and prostate cancer.

FIG. 1D is a plot showing an increase in OXPHOS following enzalutamide treatment in prostate cancer.

FIG. 1E is a plot showing an increase in OXPHOS following fulvestrant, letrozole, and palbociclib treatment in breast cancer.

FIG. 1F is a plot showing an increase in OXPHOS following cyclophosphamide treatment in breast cancer and sarcoma.

FIG. 1G is a plot showing an increase in OXPHOS following carboplatin treatment in breast cancer, endometrial cancer, non-small cell lung cancer (NSCLC), and ovarian cancer.

FIG. 1H is a plot showing an increase in OXPHOS following doxorubicin treatment in bladder cancer, breast cancer, and sarcoma.

FIG. 1I is a plot showing an increase in OXPHOS following paclitaxel treatment in breast cancer, endometrial cancer, NSCLC, and ovarian cancer.

FIG. 1J is a plot showing an increase in OXPHOS following temozolomide treatment in glioblastoma.

FIG. 2 is a plot showing that the in vitro exposure of patient-derived xenograft cells to gemcitabine sensitizes tumor cells to the compound of Formula 1.

FIG. 3 is a graph showing that the compound of Formula 1 in combination with gemcitabine prolongs animal survival.

FIG. 4A is a micrograph showing expression of pAMPK in cells that are untreated or are treated with the compound of Formula 1.

FIG. 4B is a micrograph showing expression of HIF-1a in cells that are untreated or are treated with the compound of Formula 1.

FIG. 5 describes a phase 2 clinical study protocol evaluating the use of the compound of Formula 1 in combination with the standard of care (SOC) for the treatment of BRAF-mutant metastatic melanoma.

FIG. 6 describes a phase 1b/2a clinical study protocol evaluating the use of the compound of Formula 1 in combination with the standard of care (SOC) for the treatment of pancreatic ductal adenocarcinoma.

FIG. 7A is a graph showing the response of glioblastoma patient-derived cellular subtypes to the compound of Formula 1.

FIG. 7B is a chart showing the differential response of MTC and GPM cellular subtypes to the compound of Formula 1.

FIG. 8 describes a phase 2 clinical study protocol evaluating the use of the compound of Formula 1 in the treatment of patients with glioblastoma or BRAF-mutant melanoma and brain metasteses.

FIG. 9 describes a phase 2 clinical study protocol evaluating the use of the compound of Formula 1 in combination with standard of care (SOC) for the treatment of castrate-resistant prostate cancer.

FIG. 10 is a chart describing the therapeutic window of the compound of Formula 1.

### DETAILED DESCRIPTION OF THE INVENTION

The present inventors have discovered that cancer cells that have high oxidative phosphorylation (OXPHOS) levels may survive treatment with therapeutics that target metabolic pathways other than OXPHOS, and may recur in a subject following treatment with therapeutics that do not target OXPHOS. Accordingly, the present invention provides methods of treating cancer in a subject in need thereof with OXPHOS inhibitors.

### OXPHOS Inhibitors

OXPHOS is a metabolic pathway that involves the oxidation of chemical bonds in molecules, e.g., glucose, and results in the generation of adenosine triphosphate (ATP). The method of the present invention embrace the use of all inhibitors of OXPHOS, e.g., substances that inhibit the proteins in the OXPHOS pathway, e.g., the enzymes that catalyze the chemical reactions in the OXPHOS metabolic pathway, as well as substances that inhibit the expression of the proteins in the OXPHOS metabolic pathway.

Exemplary and non-limiting OXPHOS inhibitors that may be used in accordance with the methods of the invention include biguanide compounds, e.g., metphormin, phenformin, or buformin, BAY84-243, carboxyamidotriazole (CAI), fenofibrate, meta-iodobenzylguanidine (mIBG), pyrvinium, canagliflozin, pioglitazone, rosiglitazone, amobarbital, nefazodone, αTOS, Lonidamine, Atovaquone, arsenic trioxide, nitric oxide, hydrocortisone, or an uncoupling protein (UCP), e.g., UCP1, UCP2, UCP3, UCP4, or UCP5, or a derivative or conjugate thereof.

In some embodiments, the OXPHOS inhibitor is a compound of Formula 1, N-1-pyrrolidine-N-5-(3-trifluoromethoxy)phenyl biguanide, or a pharmaceutically acceptable salt thereof.

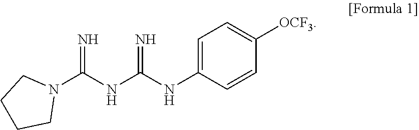 [SMILES: N=C(NC(=N)N1CCCC1)Nc1ccc(OC(F)(F)F)cc1]

Methods for synthesizing the compound of Formula 1 are disclosed in U.S. Pat. No. 9,540,325, which is incorporated by reference into the present application in its entirety.

Any suitable OXPHOS inhibitor, e.g., an OXPHOS inhibitor with an ionizable functional group, may be administered as a pharmaceutically acceptable salt, e.g., a salt of an acid selected from the group consisting of formic acid, acetic acid, propionic acid, lactic acid, butyric acid, isobutyric acid, trifluoroacetic acid, malic acid, maleic acid, malonic acid, fumaric acid, succinic acid, succinic acid monoamide, glutamic acid, tartaric acid, oxalic acid, citric acid, glycolic acid, glucuronic acid, ascorbic acid, benzoic acid, phthalic acid, salicylic acid, anthranilic acid, benzensulfonic acid, p-toluenesulfonic acid, methanesulfonic acid, dichloroacetic acid, aminooxy acetic acid, hydrochloric acid, hydrobromic acid, sulfuric acid, phosphoric acid, nitric acid, carbonic acid, and boric acid.

### Methods of Treatment

OXPHOS inhibitors may be used to treat cancer. Cancer is a group of diseases characterized by the harmful, abnormal, uncontrolled, and undesirable growth of cells. In some embodiments, the uncontrolled growth is due to cells that divide and proliferate in the absence of signals (e.g., growth factors) instructing them to do so. In some embodiments, the uncontrolled growth is due to cells which fail to respond to signals instructing them to stop growing and/or engage in programmed cell death (i.e., apoptosis). In some embodiments, cancer cells may spread throughout the body (i.e., metastasize). In some embodiments, cancer cells may form tumors. In some embodiments, cancer cells may form solid tumors.

The following description of the treatment of cancer with OXPHOS inhibitors is provided without wishing to be bound by theory. In some embodiments, cancer cells have metabolic activity, e.g., Krebs cycle and oxidative phosphorylation (OXPHOS) activity, that is unique from the metabolic activity, e.g., Krebs cycle and OXPHOS activity, of non-cancer cells. In some embodiments, OXPHOS inhibitors target the unique metabolic activity of cancer cells. In some embodiments, targeting the unique metabolic activities of cancer cells allows OXPHOS inhibitors to selectively kill cancer cells over non-cancer cells.

In some embodiments, a total population of cancer cells, e.g., the total population of cancer cells in a subject, the total population of cancer cells in an organ, the total population of cancer cells in a biological tissue, or the total population of cancer cells in a sample of cancer cells, may be composed of multiple sub-populations of cancer cells with distinct metabolic activity. One sub-population of cancer cells may rely on OXPHOS in order to satisfy their metabolic needs, while a second sub-population of cancer cells may not rely on OXPHOS in order to satisfy their metabolic needs and may instead rely on an alternative metabolic pathway, e.g., glycolysis.

In some embodiments, treatment of a population of cancer cells, e.g., a population of cancer cells in a subject, with a therapeutic that is not an OXPHOS inhibitor may target only cancer cells that do not rely on OXPHOS, and may result in the killing of only cancer cells that do not rely on OXPHOS. In some embodiments, treatment of a population of cancer cells with a therapeutic that targets a metabolic pathway other than OXPHOS may lead to the growth of cancer cells that rely on OXPHOS, e.g., a reoccurrence of OXPHOS-dependent cancer cells in a subject.

In some embodiments, the treatment of a population of cancer cells with both a therapeutic that does not target OXPHOS and a therapeutic that does target OXPHOS, i.e., an OXPHOS inhibitor, may result in the killing of both cancer cells that rely on OXPHOS and cancer cells that do not rely on OXPHOS.

In some embodiments, the therapeutic that does not target OXPHOS and the therapeutic that targets OXPHOS may be administered sequentially. In some embodiments, the therapeutic that does not target OXPHOS and the therapeutic that targets OXPHOS may be administered concurrently.

In some embodiments, an OXPHOS inhibitor may be used to treat a reoccurrence of cancer cells, e.g., a reoccurrence of OXPHOS-dependent cancer cells that survived treatment with a therapeutic that does not target OXPHOS. In some embodiments, simultaneous treatment of a population of cancer cells with a therapeutic that targets OXPHOS and a therapeutic that does not target OXPHOS results in the killing of both OXPHOS-dependent cancer cells and cancer cells that do not rely on OXPHOS. In some embodiments, simultaneous treatment of a population of cancer cells with a therapeutic that targets OXPHOS and a therapeutic that does not target OXPHOS prevents the recurrence of cancer cells, e.g., a recurrence of OXPHOS-dependent cancer cells.

Thus, OXPHOS inhibitors can be administered to a subject in an effective amount to treat cancer. Non-limiting examples of cancer that may be treated by an OXPHOS inhibitor, or a pharmaceutically acceptable salt thereof, include carcinomas, sarcomas, leukemias (e.g., an acute lymphocytic leukemia, a chronic lymphocytic leukemia, a myeloid leukemia, or a chronic myeloid leukemia), and lymphomas (e.g., a Hodgkin lymphoma or a non-Hodgkin lymphoma). In some embodiments, the cancer is a solid tumor. In some embodiments, the cancer is a metastasis. Particular types of cancer that may be treated by an OXPHOS inhibitor, or a pharmaceutically acceptable salt thereof include breast cancer, lung cancer, non-small cell lung cancer, small cell lung cancer, bladder cancer, colon cancer, rectal cancer, uterine cancer, vaginal or vulvar cancer, testicular cancer, kidney cancer, hepatic cancer, bile duct cancer, cholangiocarcinoma, gallbladder cancer, melanoma, pancreatic cancer, pancreatic ductal adenocarcinoma, BRAF-mutant metastatic melanoma, prostate cancer, castrate-resistant prostate cancer, thyroid cancer, glioblastoma, endometrial cancer, ovarian cancer, cervical cancer, brain cancer, glioblastoma, liver cancer, colorectal cancer, skin cancer, basal cell carcinoma, squamous cell carcinoma, melanoma, gynecologic cancer, head or neck cancer, mesothelioma, myeloma, esophageal cancer, and gastric cancer.

### Pancreatic Ductal Adenocarcinoma

Pancreatic ductal adenocarcinoma (PDAC) is an aggressive form of pancreatic cancer that occurs in the lining of the ducts in the pancreas. PDAC is the most common form of pancreatic cancer and accounts for more than 90% of pancreatic malignancies worldwide. PDAC is the fourth most frequent cause of cancer related deaths worldwide and has a 5-year overall survival rate of less than 8%.

### BRAF-Mutant Metastatic Melanoma

Metastatic melanoma is a form of melanoma that has spread to locations in the body other than the location where the melanoma originated. BRAF-mutant metastatic melanoma is a form of metastatic melanoma in which the gene encoding the B-Raf protein, i.e., the BRAF gene, contains one or more mutation. BRAF mutations are the most commonly found mutations in metastatic melanomas. Metastatic melanoma with BRAF mutation may be more aggressive than metastatic melanoma without BRAF mutation. BRAF-mutant metastatic melanoma may metastasize into the brain.

### Glioblastoma

Glioblastoma (GBM) is an aggressive form of cancer that may occur in the brain or spinal cord and originates in glial cells. The overall 5 year survival rate for subjects diagnosed with GBM and receiving the standard of care is less than 7%, and the average length of survival for subjects diagnosed with GBM is estimated to be 12 to 18 months.

### Castrate-Resistant Prostate Cancer

Castrate-resistant prostate cancer is a form of prostate cancer that continues to grow even when the amount of testosterone in a subject is reduced to a very low level, e.g., even after surgical or medical castration. The estimated mean survival of a subject diagnosed with castrate-resistant prostate cancer is approximately 9 to 36 months, and the quality of life for subjects diagnosed with castrate-resistant prostate cancer and receiving the standard of care is poor.

### Compositions for Combination Therapies

OXPHOS inhibitors, or a pharmaceutically acceptable salt thereof, may be administered to a subject with or without a second active component. Non-limiting examples of second active components that treat cancer that may be administered with an OXPHOS inhibitor include Lapatinib (Tykerb®), Temozolomide (Temodar®), Irinotecan (Camptosar®), Olaparib (Lynparza®), Doxorubicin (Adriamycin®), Temsirolimus (Torisel®), Gemcitabine (Gemzar®), Erlotinib (Tarceva®), Carboplatin (Paraplatin®), Paclitaxel (Taxol®), Bevcizumab (Avastin®), Ipilmumab (Yervoy®), Gefitinib (Iressa®), Apatinib, Vandetanib, (Caprelsa®), Sorafenib (Nexavar®), Dabrafenib (Tafinlar®), Vemurafenib (Zelboraf®), Fulvestrant (Faslodex®), Alpelisib (Piqray®), Palbociclib (Ibrance®), Letrozole (Femara®), Trastuzumab (Herceptin®), Pertuzumab (Perjeta®), Nelfinavir (Viracept®), Bortezomib (Velcade®), Disulfiram (Antabuse®), Ritonavir (Norvir®), Memantine hydrochloride (Namenda®), Mefloquine (Lariam®), Chloroquine, Metronomic Cyclophosphamide, Liposomal Doxorubicin (Caelyx®), Docetaxel (Taxotere), Rituximab (Rituxan®), Cyclophosphamide (Vincristine (Marqibo®), Prednisone (Rayos®), Everolimus (Zortress®), Exemestane (Aromasin®), Temsiroliumus (Torisel®), Sapanisertib, Rapamycin (Rapamune®), Trametinib (Mekinist®), Sintilimab (Tyvyt®), Nivolumab (Opdivo®), Durvalumab (Imfinzi®), Pembrolizumab (Keytruda®), or Rosiglitazone (Avandia®).

### Pharmaceutical Compositions

The administration of OXPHOS inhibitors may be by any suitable means that results in treatment of cancer. OXPHOS inhibitors may be contained in any appropriate amount in any suitable carrier substance and is generally present in an amount of 1-95% by weight of the total weight of the composition. The composition may be provided in a dosage form that is suitable for the sublingual, buccal, oral, parenteral (e.g., intravenously, intramuscularly), pulmonary, intranasal, transdermal, vaginal, or rectal administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, sprays, vapors, or aerosols. The pharmaceutical compositions may be formulated according to conventional pharmaceutical practice (see, e.g., Remington: The Science and Practice of Pharmacy, (23rd ed.) ed. A. Adejare., 2020, Academic Press, Philadelphia, PA).

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the active compound within the body over an extended period of time; (ii) formulations that after a predetermined lag time create a substantially constant concentration of the active compound within the body over an extended period of time; and (iii) formulations that sustain active compound action during a predetermined time period by maintaining a relatively, constant, effective active compound level in the body with concomitant minimization of undesirable side effects associated with fluctuations in the plasma level of the active compound (sawtooth kinetic pattern).

Any one of a number of strategies can be pursued in order to obtain controlled release of the active compound in question. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the active compound in a controlled manner. Examples include single or multiple unit tablet or capsule compositions, oil solutions, suspensions, emulsions, microcapsules, microspheres, nanoparticles, patches, and liposomes.

The pharmaceutical compositions contemplated by the invention may an OXPHOS inhibitor, or a pharmaceutically acceptable salt thereof, in a mixture with non-toxic pharmaceutically acceptable excipients. Pharmaceutically acceptable excipients are known to the skilled artisan. Excipients may be, for example, inert diluents or fillers such as sucrose, sorbitol, sugar, mannitol, microcrystalline cellulose, in particular microcrystalline cellulose PH101 or microcrystalline cellulose PH200, starches including potato starch, calcium carbonate, sodium chloride, lactose, calcium phosphate, calcium sulfate, or sodium phosphate; disintegrants such as crospovidone, sodium alginate, colloidal magnesium, aluminum silicate, calcium silicate, sodium starch glycolate, acrylic acid derivatives, microcrystalline cellulose, sodium carboxymethyl cellulose, calcium carboxymethyl cellulose, modified cellulose gum, cross-linked povidone, alginic acid and alginates, pregelatinised starch, modified corn starch cellulose derivatives including microcrystalline cellulose, starches including potato starch, croscarmellose sodium, alginates, or alginic acid; binders such as sucrose, glucose, sorbitol, acacia, alginic acid, gelatin, starch, pregelatinized starch, microcrystalline cellulose, magnesium aluminum silicate, carboxymethylcellulose sodium, methylcellulose, hydroxypropyl methylcellulose, hydroxypropyl cellulose EXF, ethylcellulose, polyvinylpyrrolidone, or polyethylene glycol; lubricants and/or glidants such as colloidal silicon dioxide, particularly colloidal silicon dioxide Cab-O-Sil® M5P, glycerol tribehenate, magnesium stearate, calcium stearate, talc, sodium stearyl fumarate, sodium behenate, stearic acid, cetyl alcohol, polyoxyethylene glycol, leucine, sodium benzoate, stearates, polyethylene glycol, glyceryl monostearate, glyceryl palmitostearate, liquid paraffin, poloxamer, sodium lauryl sulphate, magnesium lauryl sulphate, hydrogenated castor colloidal silicon dioxide, palmitostearate, stearic acid, zinc stearate, stearyl alcohol, silicas, or hydrogenated vegetable oil; anti-caking agents such as colloidal silicon dioxide, microcrystalline cellulose, tricalcium phosphate, microcrystalline cellulose, magnesium stearate, sodium bicarbonate, sodium ferrocyanide, potassium ferrocyanide, calcium ferrocyanide, calcium phosphate, sodium silicate, colloidal silicon dioxide, calcium silicate, magnesium trisilicate, talcum powder, sodium aluminosilicate, potassium aluminum silicate, calcium aluminosilicate, bentonite, aluminum silicate, stearic acid, polydimethylsiloxane. Other pharmaceutically acceptable excipients may be colorants, flavoring agents, plasticizers, humectants, and buffering agents.

### Dosages

The dosage of the OXPHOS inhibitors used in the methods described herein, e.g., the OXPHOS inhibitor having the structure of Formula I, can vary depending on many factors, e.g., the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The composition used in the methods described herein may be administered initially in a suitable dosage that may be adjusted as required, depending on the clinical response. In general, a suitable daily dose of a compound of the invention will be that amount of the compound that is the lowest dose effective to produce a therapeutic effect. Such an effective dose will generally depend upon the factors described above.

While the attending physician ultimately will decide the appropriate amount and dosage regimen, an effective amount of a composition of the invention may be determined by the weight of the subject. For example, an effective amount may be between about 8 mg once a day (QD) and about 6,400 mg QD, e.g., between about 8 mg QD and about 3,200 mg QD, between about 8 mg QD and about 3,200 mg QD, between about 8 mg QD and about 1,200 mg QD, between about 8 mg QD and about 800 mg QD, e.g., between about 16 mg QD and about 400 mg QD, between about 32 mg QD and about 200 mg QD, between about 64 mg QD and 100 mg QD, between about 100 mg QD and 400 mg QD, between about 200 mg QD and about 300 mg QD, between about 400 mg QD and about 1,200 mg QD, between about 600 mg QD and about 1,000 mg QD, between about 700 mg QD and about 900 mg QD, between about 750 mg QD and about 850 mg QD, of a solid dosage form or composition described herein. In some embodiments, an effective amount may be between 8 mg QD±0.8 mg QD and 6,400 mg QD±640 mg QD, 8 mg QD±0.8 mg QD and 3,200 mg QD±320 mg QD, 8 mg QD±0.8 mg QD and 2,400 mg QD±240 mg QD, 8 mg QD±0.8 mg QD and 1,200 mg QD±160 mg QD, 8 mg QD±0.8 mg QD and 800 mg QD±80 mg QD, between 16 mg QD±1.6 mg QD and 400 mg QD±40 mg QD, between 32 mg QD±3.2 mg QD and 200 mg QD±20 mg QD, between 64 mg QD±6.4 mg QD and 100 mg QD±10 mg QD, between 100 mg QD±10 mg QD and 400 mg QD±40 mg QD, between 200 mg QD±20 mg QD and 300 mg QD±30 mg QD, between 400 mg QD±40 mg and 1,200 mg QD±120 mg, between 600 mg QD±60 mg and 1,000 mg QD±100 mg, between 700 mg QD±70 mg and 900 mg QD±90 mg, between 750 mg QD±75 mg and 850 mg QD±80 mg of a solid dosage form or composition described herein. In some embodiments, an effective amount may be between 8 mg QD and 6,400 mg QD, between 8 mg QD and 3,200 mg QD, between 8 mg QD and 2,400 mg QD, 8 mg QD and 1,200 mg QD, between 8 mg QD and 800 mg QD, between 16 mg QD and 400 mg QD, between 32 mg QD and 200 mg QD, between 64 mg QD and 100 mg QD, between 100 mg QD and 400 mg QD, between 200 mg QD and 300 mg QD, between 400 mg QD and 1,200 mg QD, between 600 mg QD and 1,000 mg QD, between 700 mg QD and 900 mg QD, between 750 mg QD and 850 mg QD of a solid dosage form or composition described herein.

In some embodiments, it may be a dose of about 8 mg QD of a solid dosage form or composition described herein. In some embodiments, it may be a dose of about 16 mg QD of a solid dosage form or composition described herein. In some embodiments, it may be a dose of about 32 mg QD of a solid dosage form or composition described herein. In some embodiments, it may be a dose of about 64 mg QD of a solid dosage form or composition described herein. In some embodiments, it may be a dose of about 100 mg QD of a solid dosage form or composition described herein. In some embodiments, it may be a dose of about 200 mg QD of a solid dosage form or composition described herein. In some embodiments, it may be a dose of about 300 mg QD of a solid dosage form or composition described herein. In some embodiments, it may be a dose of about 400 mg QD of a solid dosage form or composition described herein. In some embodiments, it may be a dose of about 800 mg QD of a solid dosage form or composition described herein. In some embodiments, it may be a dose of about 1,200 mg QD of a solid dosage form or composition described herein. In some embodiments, it may be a dose of about 2,400 mg QD of a solid dosage form or composition described herein. In some embodiments, it may be a dose of about 3,200 mg QD of a solid dosage form or composition described herein. In some embodiments, it may be a dose of about 6,400 mg QD of a solid dosage form or composition described herein.

In some embodiments, it may be a dosage of 8 mg QD±0.8 mg QD of a solid dosage form or composition described herein. In some embodiments, it may be a dosage of 16 mg QD±1.6 mg QD of a solid dosage form or composition described herein. In some embodiments, it may be a dosage of 32 mg QD±3.2 mg QD of a solid dosage form or composition described herein. In some embodiments, it may be a dosage of 64 mg QD±6.4 mg QD of a solid dosage form or composition described herein. In some embodiments, it may be a dosage of 100 mg QD±10 mg QD of a solid dosage form or composition described herein. In some embodiments, it may be a dosage of 200 mg QD±20 mg QD of a solid dosage form or composition described herein. In some embodiments, it may be a dosage of 300 mg QD±30 mg QD of a solid dosage form or composition described herein. In some embodiments, it may be a dosage of 400 mg QD±40 mg QD of a solid dosage form or composition described herein. In some embodiments, it may be a dosage of 800 mg QD±80 mg QD of a solid dosage form or composition described herein. In some embodiments, it may be a daily dosage of 1,200 mg QD±120 mg of a solid dosage form or composition described herein. In some embodiments, it may be a daily dosage of 2,400 mg QD±240 mg of a solid dosage form or composition described herein. In some embodiments, it may be a daily dosage of 3,200 mg±320 mg of a solid dosage form or composition described herein. In some embodiments, it may be a daily dosage of 6,400 mg±640 mg of a solid dosage form or composition described herein.

In some embodiments, it may be a dosage of 8 mg QD of a solid dosage form or composition described herein. In some embodiments, it may be a dosage of 16 mg QD of a solid dosage form or composition described herein. In some embodiments, it may be a dosage of 32 mg QD of a solid dosage form or composition described herein. In some embodiments, it may be a dosage of 64 mg QD of a solid dosage form or composition described herein. In some embodiments, it may be a dosage of 100 mg QD of a solid dosage form or composition described herein. In some embodiments, it may be a dosage of 200 mg QD of a solid dosage form or composition described herein. In some embodiments, it may be a dosage of 300 mg QD of a solid dosage form or composition described herein. In some embodiments, it may be a dosage of 400 mg QD of a solid dosage form or composition described herein. In some embodiments, it may be a dosage of 800 mg QD of a solid dosage form or composition described herein. In some embodiments, it may be a dosage of 1,200 mg QD of a solid dosage form or composition described herein. In some embodiments, it may be a dosage of 2400 mg QD of a solid dosage form or composition described herein. In some embodiments, it may be a dosage of 3200 mg QD of a solid dosage form or composition described herein. In some embodiments, it may be a dosage of 6400 mg QD of a solid dosage form or composition described herein.

In some embodiments, it may be a daily dosage of about 1 mg/kg to about 80 mg/kg of a solid dosage form or composition described herein. In some embodiments, it may be a daily dosage of about 1 mg/kg to about 40 mg/kg of a solid dosage form or composition described herein. In some embodiments, it may be a daily dosage of about 3 mg/kg to 30 mg/kg of a solid dosage form or composition described herein. In some embodiments, it may be a daily dosage of about 5 mg/kg to about 25 mg/kg of a solid dosage form or composition described herein. In some embodiments, it may be a daily dosage of about 10 mg/kg to about 20 mg/kg of a solid dosage form or composition described herein. In some embodiments, it may be a daily dosage of about 12 mg/kg to about 18 mg/kg of a solid dosage form or composition described herein. In some embodiments, it may be a daily dosage of about 1.25 mg/kg to about 15 mg/kg of a solid dosage form or composition described herein. In some embodiments, it may be a daily dosage of about 2.5 mg/kg to about 10 mg/kg of a solid dosage form or composition described herein. In some embodiments, it may be a daily dosage of about 5 mg/kg to about 8 mg/kg of a solid dosage form or composition described herein. In some embodiments, it may be a daily dosage of 1 mg/kg±0.1 mg/kg to 80 mg/kg±8300.0 mg/kg of a solid dosage form or composition described herein. In some embodiments, it may be a daily dosage of 1 mg/kg±0.1 mg/kg to 40 mg/kg±4.0 mg/kg of a solid dosage form or composition described herein. In some embodiments, it may be a daily dosage of 3 mg/kg±0.3 mg/kg of a solid dosage form or composition described herein to 30 mg/kg±3 mg/kg of a solid dosage form or composition described herein. In some embodiments, it may be a daily dosage of 5 mg/kg±0.5 mg/kg to about 25±2.5 mg/kg of a solid dosage form or composition described herein. In some embodiments, it may be a daily dosage of 10 mg/kg±1.0 mg/kg to about 20±2 mg/kg of a solid dosage form or composition described herein. In some embodiments, it may be a daily dosage of 12 mg/kg±1.2 mg/kg to 18 mg/kg±1.8 mg/kg of a solid dosage form or composition described herein. In some embodiments, it may be a daily dosage of 1.25±0.125 mg/kg to 15±1.5 mg/kg of a solid dosage form or composition described herein. In some embodiments, it may be a daily dosage of 2.5 mg/kg±0.25 to 10±1.0 mg/kg of a solid dosage form or composition described herein. In some embodiments, it may be a daily dosage of 5±0.5 mg/kg to 8±0.8 mg/kg of a solid dosage form or composition described herein. In some embodiments, it may be a daily dosage of 1 mg/kg to 40 mg/kg of a solid dosage form or composition described herein. In some embodiments, it may be a daily dosage of 3 mg/kg to 30 mg/kg of a solid dosage form or composition described herein. In some embodiments, it may be a daily dosage of 5 mg/kg to 25 mg/kg of a solid dosage form or composition described herein. In some embodiments, it may be a daily dosage of 10 mg/kg to 20 mg/kg of a solid dosage form or composition described herein. In some embodiments, it may be a daily dosage of 12 mg/kg to 18 mg/kg of a solid dosage form or composition described herein. In some embodiments, it may be a daily dosage of 1.25 mg/kg to 15 mg/kg of a solid dosage form or composition described herein. In some embodiments, it may be a daily dosage of 2.5 mg/kg to 10 mg/kg of a solid dosage form or composition described herein. In some embodiments, it may be a daily dosage of 5 mg/kg to 8 mg/kg of a solid dosage form or composition described herein. In some embodiments, it may be a daily dosage of about 30 mg/kg. In some embodiments, it may be a daily dose of about 50 mg/kg. In some embodiments, it may be a daily dose of about 60 mg/kg.

### Release Profile

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings (e.g., enteric coatings). Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above.

Multiple techniques for applying enteric coatings to pharmaceutical compositions are known in the art. In one non-limiting example, tablets and/or capsules may undergo a drum coating process in which they are tumbled in a cylindrical drum while being sprayed by a coating solution. In an additional non-limiting example, tablets and/or capsules may undergo a fluid-bed coating process in which air is passed over tablets or capsules at a sufficient velocity to separate the tables and/or capsules into individual units, at which point the tablets/capsules are sprayed with coating solution from above (e.g., top spray coating), from below (e.g., bottom spray coating), or from the side (e.g., HP spray coating). The amount of coating is determined by the amount of solution sprayed. The coating may be applied in a single application or may be built up in layers through the use of multiple applications.

### Solid Dosage Forms for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These compositions can be prepared in a variety of ways well known in the pharmaceutical art, and can be made so as to release OXPHOS inhibitors, e.g., the compound of Formula 1, or a pharmaceutically acceptable salt thereof, in specific segments of the gastrointestinal tract at controlled times by a variety of excipients and formulation technologies. For example, formulations may be tailored to address a specific disease, to achieve plasma levels of OXPHOS inhibitors, e.g., the compound of Formula 1, required to achieve therapeutic efficacy, to enable a desired duration of drug effect, and to provide a set of compositions with varying drug release.

### OXPHOS Inhibition

Multiple techniques for measuring OXPHOS inhibition are known in the art. Exemplary and non-limiting methods for measuring OXPHOS inhibition are described below.

### Measurement of Mitochondrial Complex I Inhibition

The electron transfer complex in mitochondria is composed of 5 complexes. The complex I accept electrons from NADH produced from glycolysis and TCA cycle and the electrons move to complex II, III and IV and the electron is finally transferred to O₂ and water molecule is generated. During the electron transfer, proton gradient is generated and the chemical gradient is a driving source to synthesize ATP at complex V. The mitochondrial inhibition of complex I indirectly assessed by measuring the oxygen consumption rate (OCR) at complex IV. When the mitochondria ETC is inhibited, glycolysis is up-regulated and lactate production is increased. The solution outside of cells becomes acidic (lower pH) as lactate is transported to outside of cells. OCR and Extracellular acidification rate (ECAR) are determined by XF Analyzer (Seahorse Biosciences). Thus, the OXPHOS inhibitory effect of a compound on a sample of cells such as cancer cells, e.g., the OXPHOS inhibitory effect caused by the compound of Formula I, can be measured by measuring the oxygen consumption rate of a sample of cells of the extracellular acidification rate of a sample of cells.

### Cytotoxicity Assay in Low Glucose Condition

The inhibition of oxidative phosphorylation (OXPHOS) is not cytotoxic to cells in normal glucose condition, because it is postulated that normal: cells have compensatory mechanism under energy stress conditions such as low glucose. However OXPHOS inhibitors show cytotoxic effect on cells in the glucose deprived condition. The glucose deprived condition is observed in tumor microenvironment potentially due to poor angiogenesis. Therefore the OXPHOS inhibitors may show anti-cancer effect on cancer cells in low glucose condition that may depict tumor microenvironment.

### EQUIVALENTS AND SCOPE

Those skilled in the art will recognize or be able to ascertain using no more than routine experimentation, many equivalents to the specific embodiments in accordance with the invention described herein. The scope of the present invention is not intended to be limited to the above Description.

The following Examples are illustrative only and not intended to limit the invention in any way.

### EXAMPLES

### Example 1. Pre- and Post-Treatment Biopsies of Patients Receiving Cancer Therapies

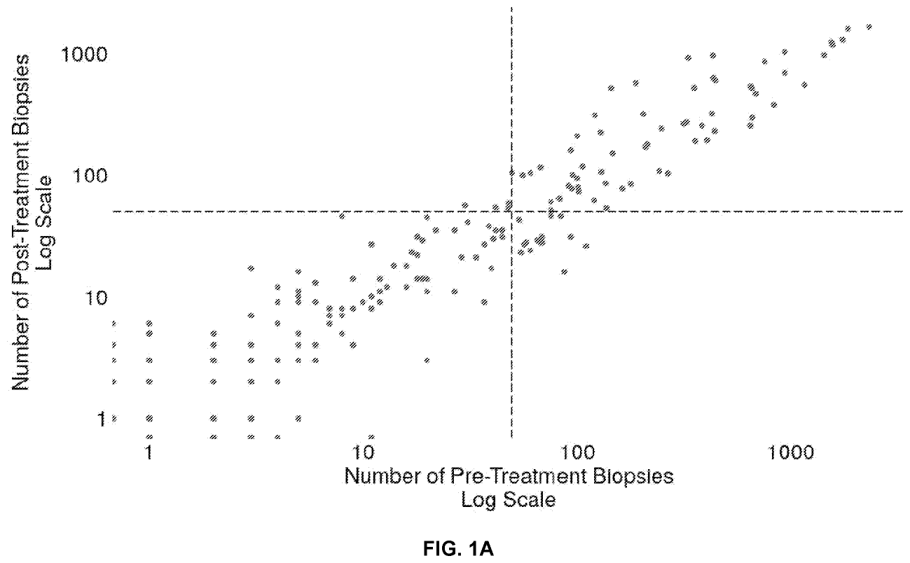

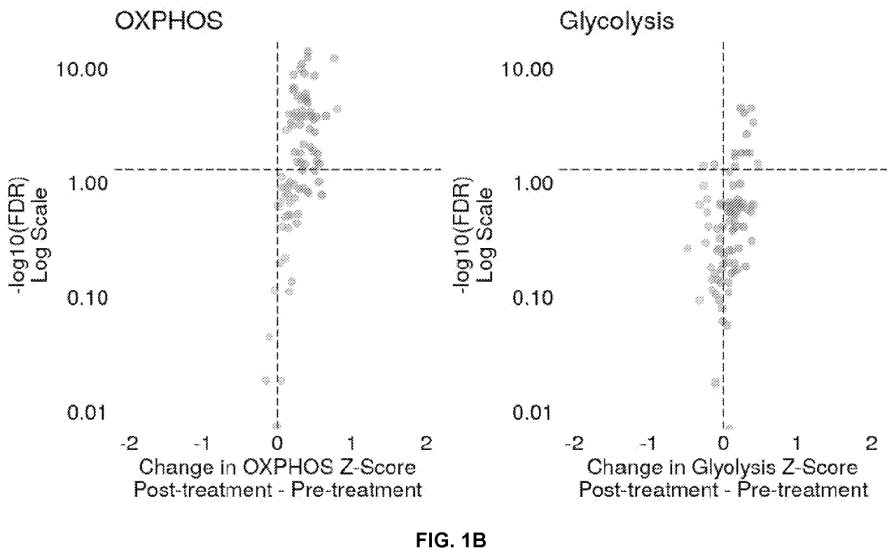

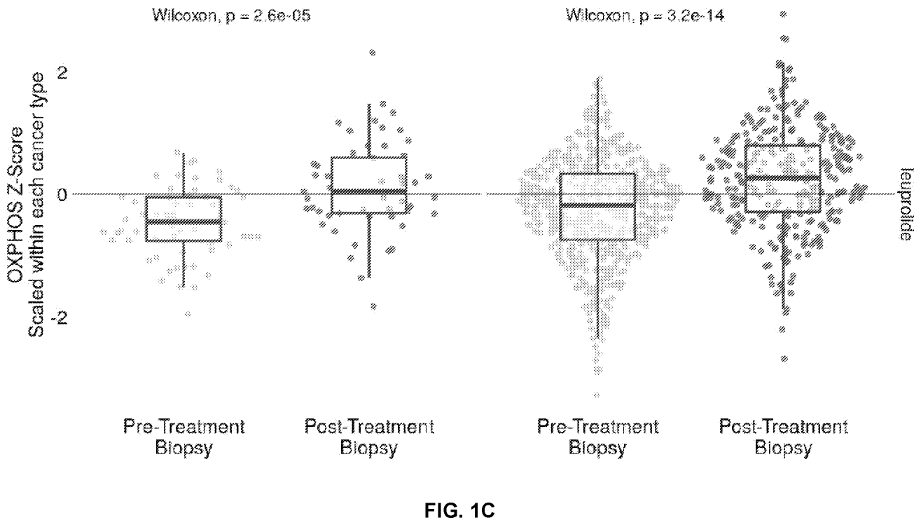

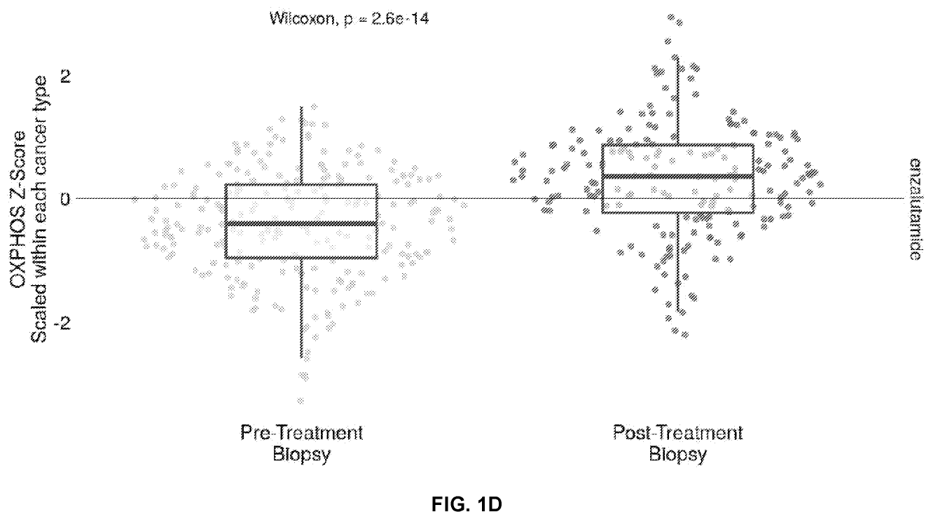

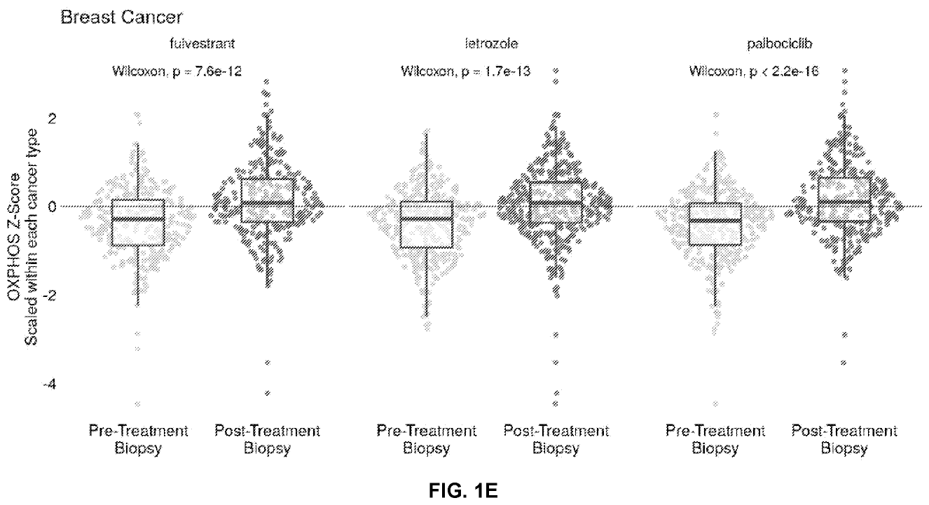

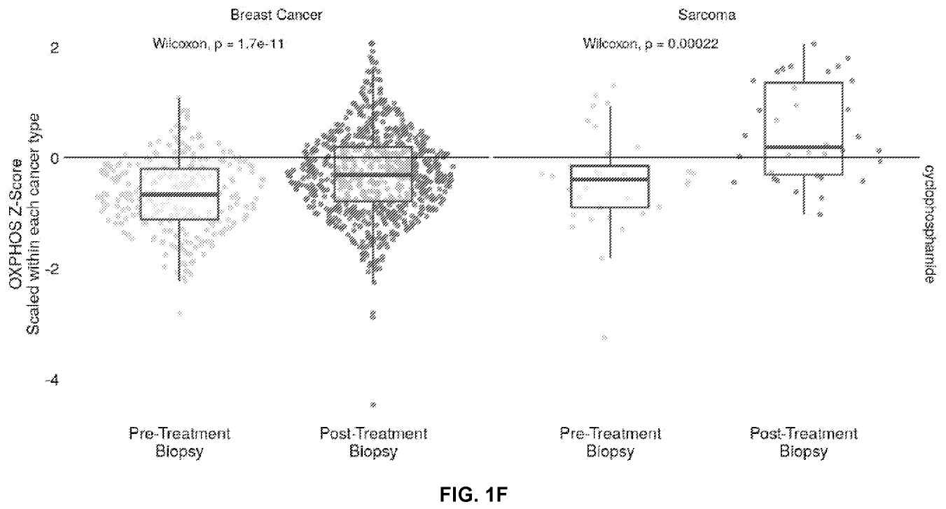

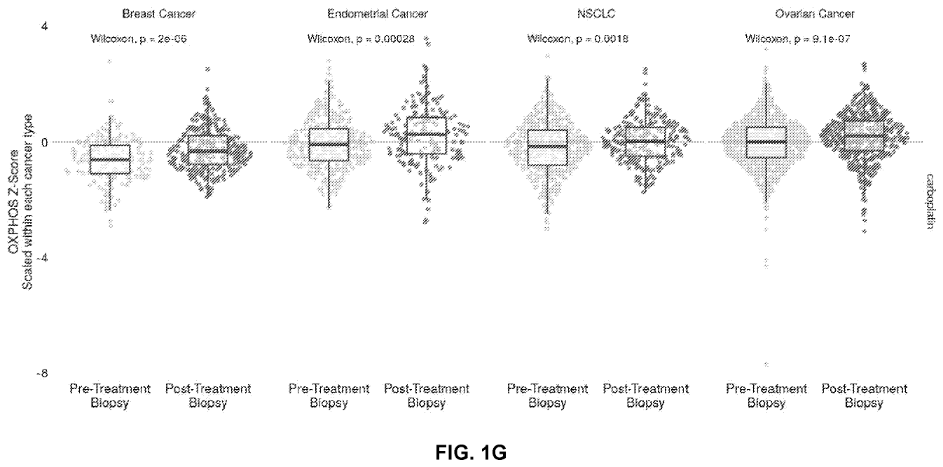

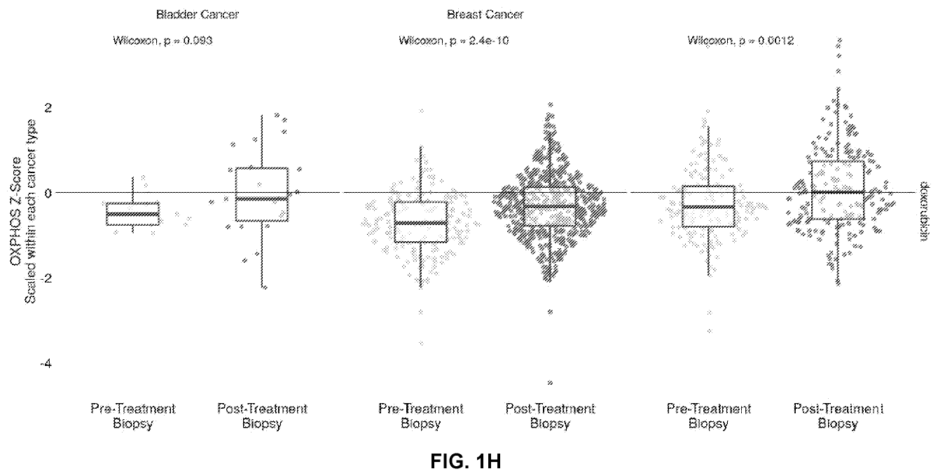

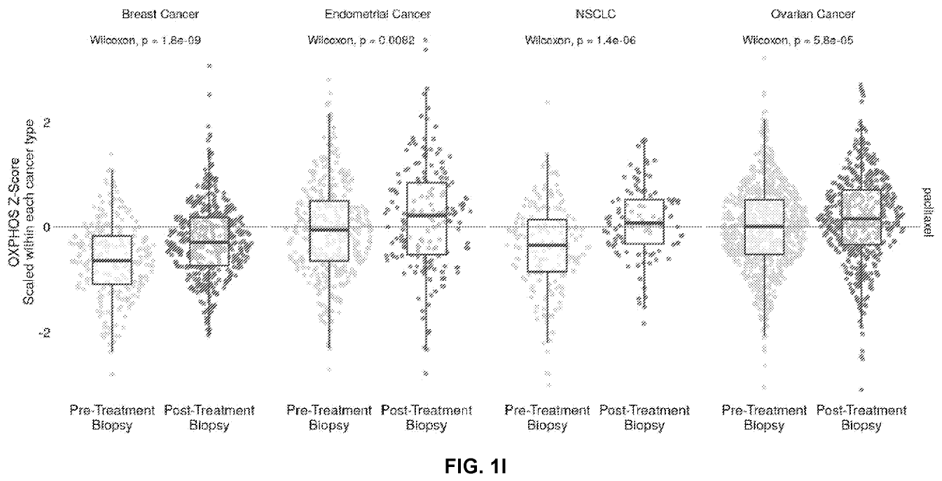

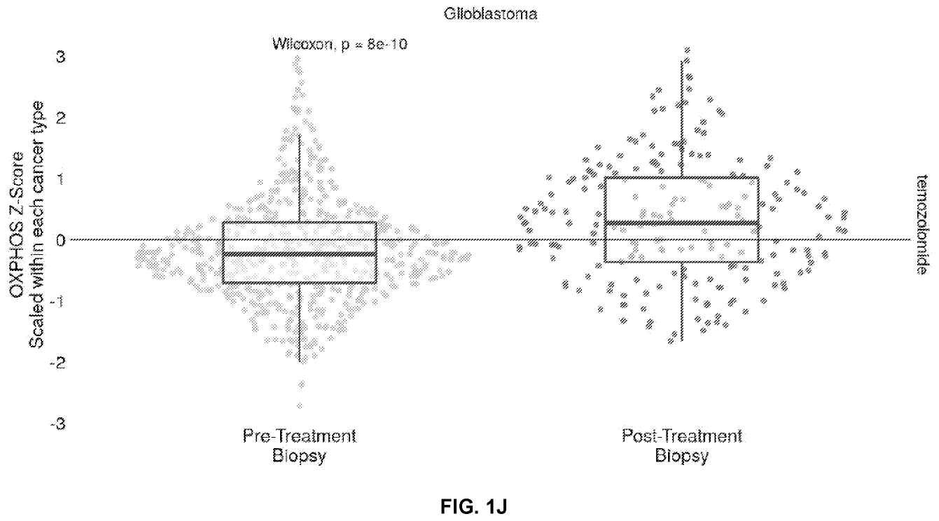

Biopsies were collected from subjects undergoing treatment for cancer (FIG. 1A). Cells captured in post-treatment biopsies utilized OXPHOS more than cells captured in pre-treatment biopsies, and utilized glycolysis less than cells captured in pre-treatment biopsies (FIG. 1B-1J). It was found that:
1) ENO1 and SLC45A1 expression are not robust biomarkers of OXPHOS, each showing positive and negative correlations in different cancer types.
a) Single-copy loss of the locus (LOH) is associated with higher OXPHOS levels, especially in cholangiocarcinoma where it is prevalent (˜33%)
2) Pan-cancer gene based analyses identified 7 genes with higher pathogenic mutation burden associated with increased OxPhos pathway scores (FDR<5%)—STK11, CDH1, PRKAR1A, KEAP1, TSC2, MAP3K1 and PIK3CA
3) Single variant analyses tested within each cancer identified 7 variants associated with increased OxPhos scores (FDR<10%) with moderate effect sizes—AKT1, TP53 (2 variants) STAG2, PIK3CA, KMT2C, and MTOR
4) Single variant meta-analyses across cancers identified 2 variants in genes RET and TP53 with contributions detected in more than once cancer
5) Most therapies appear to increase OXPHOS (or select for clones with high OXPHOS levels)
a) In particular, leuprolide, enzalutamide, fulvestrant, letrozole, palbociclib, cyclophosphamide, carboplatin, doxorubicin, paclitaxel, and temozolomide were the most significant therapies.

### Example 2. Evaluation of the Combination of Formula 1 and Gemcitabine on Treating Pancreatic Ductal Adenocarcinoma

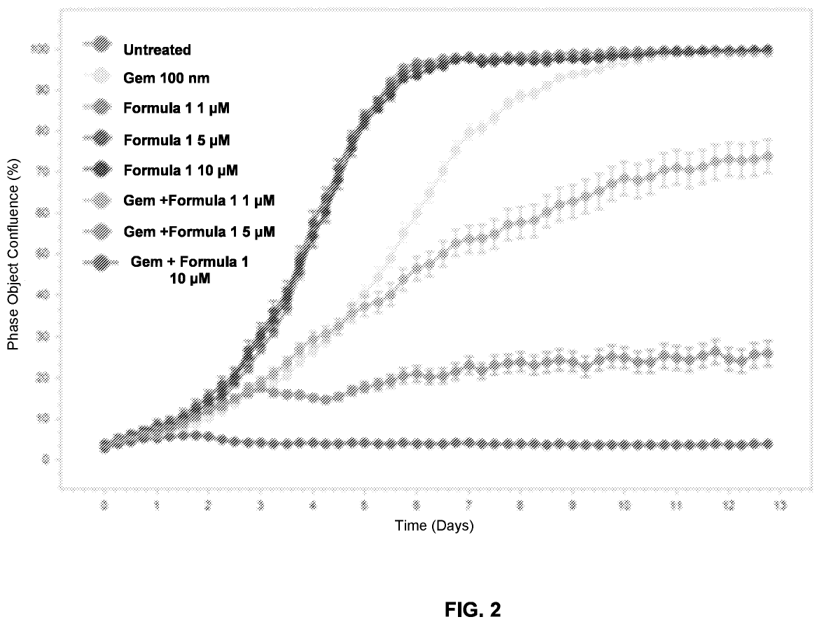

Patient derived xenograft cells were treated with the compound of Formula 1, Gemcitabine (Gem), or the compound of Formula 1 and gemcitabine together (FIG. 2). Cells treated with both Gem and Formula 1 grew slower than cells treated with Formula 1 or Gem alone.

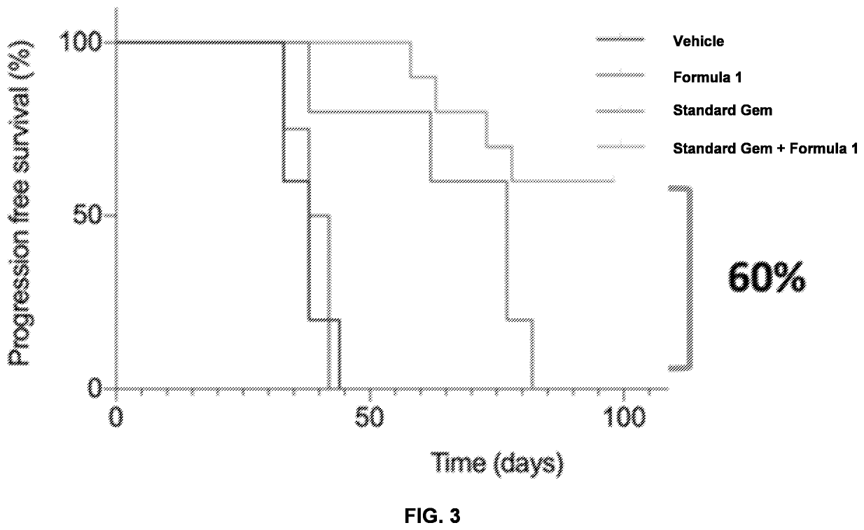

Animals with pancreatic ductal adenocarcinoma were treated with either the compound of Formula 1, Gem, or a combination of the compound of Formula 1 and Gem (FIG. 3). The animals treated with Gem and the compound of Formula 1 combined survived longer than the animals treated with either Gem or the compound of Formula 1 alone.

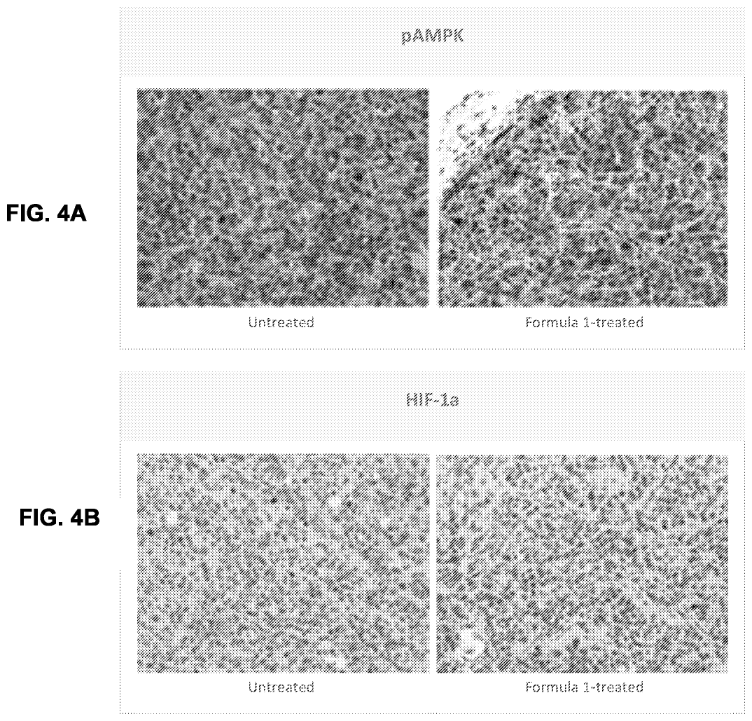

Expression of mechanism of action biomarkers pAMPK and HIF-1a inuntreated cells versus cells treated with the compound of Formula 1 support that Gem in combination with compound 1 inhibits cancer cell growth and prolongs survival (FIGS. 4a and 4b).

### Example 3. Clinical Study of Formula 1 in Combination with the Standard of Care for Treating BRAF-Mutant Metastatic Melanoma in Human Subjects

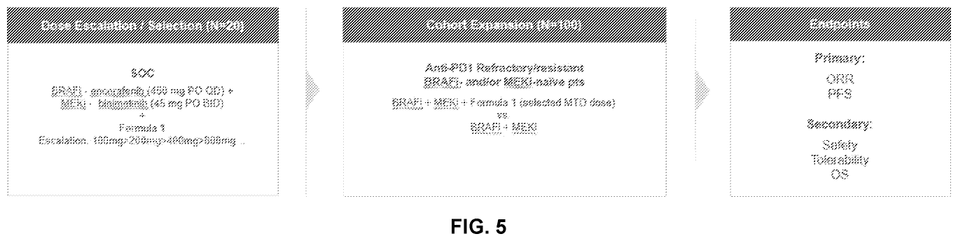

Subjects with BRAF-mutant metastatic melanoma are enrolled in a phase 2 clinical study to evaluate the effectiveness of the compound of Formula 1 in combination with the standard of care (SOC) for treating BRAF-mutant Metastatic Melanoma (FIG. 5). Dose escalation and dose selection is determined with an initial cohort of 20 subjects that are administered the SOC, which is BRAFi (encorafenib, 450 mg PO QD) and MEKi (binimetinib (45 mg PO BID), in combination with the compound of Formula 1 (escalating doses from 100 mg to 200 mg to 400 mg to 800 mg).

Following the determination of the dose of the compound of Formula 1, the cohort is expanded to 100 subjects. One arm is administered BRAFi, MEKi, and the compound of Formula 1, and the second arm is administered BRAFi and MEIKi.

The primary endpoints are ORR and PFS. The secondary endpoints are safety, tolerability, and OS.

### Example 4. Clinical Study of Formula 1 in Combination with the Standard of Care for Treating Pancreatic Ductal Adenocarcinoma in Human Subjects

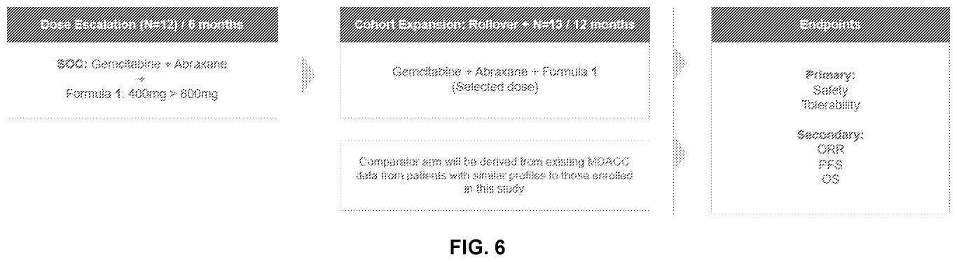

Subjects with pancreatic ductal adenocarcinoma are enrolled in a clinical study to evaluate the effectiveness of the compound of Formula 1 in combination with the standard of care (SOC) for treating pancreatic ductal adenocarcinoma (FIG. 6). Dose escalation and dose selection is determined with an initial cohort of 12 subjects who are administered SOC, which is gemcitabine and abraxane, in combination with the compound of Formula 1 (400 mg>800 mg).

Following determination of the dose of the compound of Formula 1, the cohort is expanded to 13 subjects. One arm is administered SOC+the compound of Formula 1 at the determined dose. The comparator arm is derived from existing MDACC data from patients with similar profiles to those enrolled in this study.

The primary endpoints are safety and tolerability. The secondary endpoints are ORR, PFS, and OS.

### Example 5. Evaluation of the Response of Glioblastoma Patient-Derived Cellular Subtypes to the Compound of Formula 1

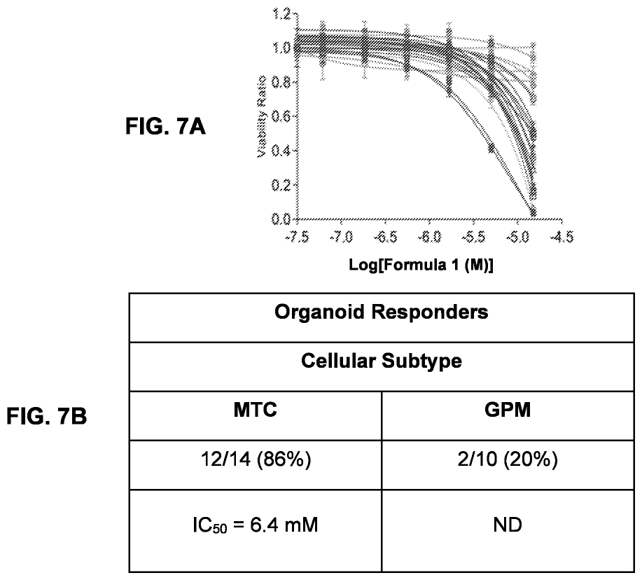

Glioblastoma patient-derived cellular subtypes were treated with the compound of Formula 1 (FIG. 7A). The MTC GBM cellular subtype was more responsive to treatment with the compound of Formula 1 than the GPM cellular subtype (FIG. 7B).

### Example 6. Clinical Study of the Compound of Formula 1 in Combination with the Standard of Care for Treating BRAF-Mutant Metastatic Melanoma in Human Subjects

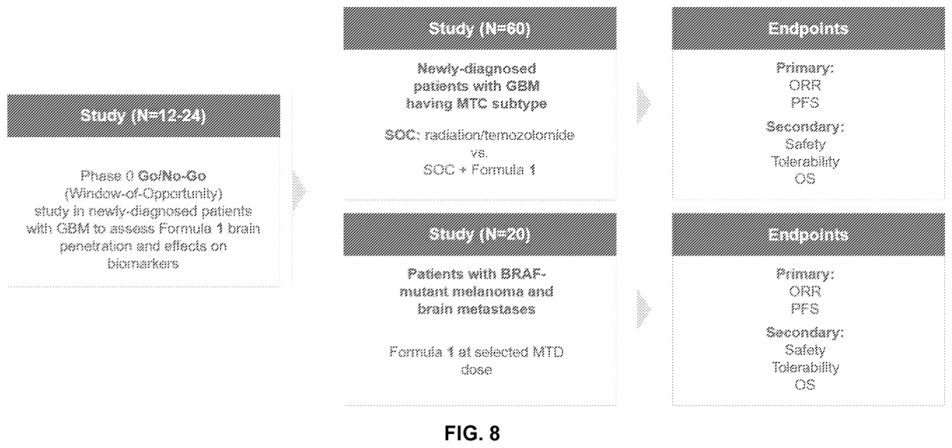

A phase 0 go/no-go (window of opportunity) study in newly-diagnosed subjects with GBM to assess the compound of Formula 1 brain penetration and effects on biomarkers is conducted with 12-24 subjects (FIG. 8).

Newly diagnosed subjects with GBM (N=60) having the MTC subtype are enrolled in a study to evaluate the effectiveness of the compound of Formula 1 in combination with the SOC, which is radiation/tezolomide, in treating GBM with MTC subtype. One arm is administered the SOC alone, and a second arm is administered SOC in combination with the compound of Formula 1.

Subjects with BRAF-mutant melanoma and brain metastases (N=20) are enrolled in a study to evaluate the effectiveness of the compound of Formula 1 in treating BRAF-mutant melanoma and brain metastases. The subjects are administered the compound of Formula 1 at the selected MTD dose.

The primary endpoints for both the study of subjects with GBM having MTC subtype and the study of patients with BRAF-mutant melanoma and brain metastases are ORR and OFS. The secondary endpoints are safety, tolerability, and OS.

### Example 7. Clinical Study of Formula 1 in Combination with the Standard of Care for Treating Castrate-Resistant Prostate Cancer

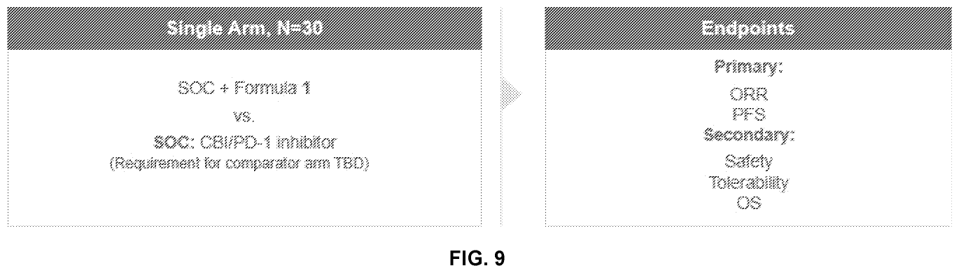

Subjects (N=30) are enrolled in a clinical study to evaluate the effectiveness of the compound of Formula 1 in combination with the SOC, which is CBI/PD-1 inhibitor, in treating castrate-resistant prostate cancer (FIG. 9). One arm is administered the SOC+the compound of Formula 1. One arm is administered the SOC alone. The primary endpoints are ORR and PFS. The secondary endpoints are safety, tolerability, and OS.

### Example 8. Therapeutic Window of the Compound of Formula 1

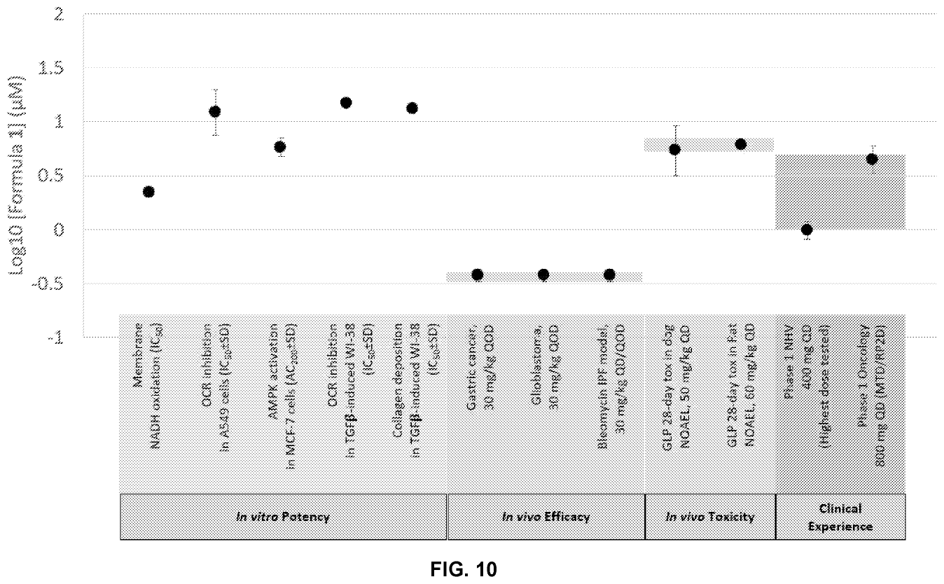

The therapeutic window of the compound of Formula 1 was determined for various indications (FIG. 10).

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

All references, patents, patent application publications, and patent applications recited herein ae hereby incorporated by reference in their entirety.

Other embodiments are in the claims.

What is claimed is:

1. A method of treating a cancer in a subject, the method comprising administering an oxidative phosphorylation (OXPHOS) inhibitor to the subject, wherein the cancer is refractory to a first line of treatment or is recurrent.

2. The method of claim 1, wherein the first line of treatment is the standard of care for the cancer.

3. The method of claim 1 or 2, wherein the cancer is a carcinoma, a sarcoma, a leukemia, or a lymphoma.

4. The method of any one of claim 1-3, wherein the cancer is a solid tumor.

5. The method of any one of claims 1-4, wherein the cancer is a metastatic cancer.

6. The method of claim 3, wherein the leukemia is an acute lymphocytic leukemia, a chronic lymphocytic leukemia, a myeloid leukemia, or a chronic myeloid leukemia.

7. The method of claim 3, wherein the lymphoma is a Hodgkin lymphoma or a non-Hodgkin lymphoma.

8. The method of claim 3, wherein the cancer is a breast cancer, lung cancer, non-small cell lung cancer, small cell lung cancer, bladder cancer, colon cancer, rectal cancer, uterine cancer, vaginal or vulvar cancer, testicular cancer, kidney cancer, hepatic cancer, bile duct cancer, cholangiocarcinoma, gallbladder cancer, melanoma, pancreatic cancer, pancreatic ductal adenocarcinoma, BRAF-mutant metastatic melanoma, prostate cancer, castrate-resistant prostate cancer, thyroid cancer, glioblastoma, endometrial cancer, ovarian cancer, cervical cancer, brain cancer, glioblastoma, liver cancer, colorectal cancer, skin cancer, basal cell carcinoma, squamous cell carcinoma, melanoma, gynecologic cancer, head or neck cancer, mesothelioma, myeloma, esophageal cancer, or gastric cancer.

9. The method of any one of claims 1-8, wherein the OXPHOS inhibitor is a compound of Formula 1 below or a pharmaceutically acceptable salt thereof

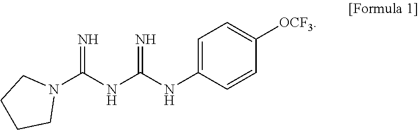 [SMILES: N=C(NC(=N)N1CCCC1)Nc1ccc(OC(F)(F)F)cc1]

10. The method of claim 9, wherein the compound of Formula 1 is formulated as a tablet or a capsule.

11. The method of claim 9, wherein the tablet or capsule is formulated for delayed release.

12. The method of claim 10 or 11, wherein the tablet or capsule comprises an enteric coating.

13. The method of any one of claims 9-12, wherein the compound of Formula 1 is administered at a dose of about 100 mg, 200 mg, 400 mg, 800 mg, or 1200 mg per day.

14. The method of claim 13, wherein the compound of Formula 1 is administered at a dose of about 800 mg per day.

15. The method of any one of claims 1-9, wherein the OXPHOS inhibitor is administered in combination with a second therapeutic compound.

16. A method of treating pancreatic ductal adenocarcinoma (PDAC), the method comprising administering a pharmaceutical composition to a subject in need thereof with a therapeutically effective dose, wherein the composition comprises a compound of Formula 1 below or a pharmaceutically acceptable salt thereof as an active ingredient

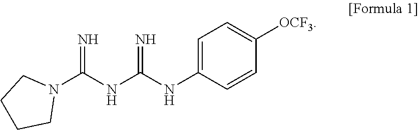 [SMILES: N=C(NC(=N)N1CCCC1)Nc1ccc(OC(F)(F)F)cc1]

17. The method of claim 16, wherein the compound of Formula 1 is administered in combination with Gemcitabine and Abraxane.

18. A method of treating BRAF-mutant melanoma, the method comprising administering a pharmaceutical composition to a subject in need thereof with a therapeutically effective dose, wherein the composition comprises a compound of Formula 1 below or a pharmaceutically acceptable salt thereof as an active ingredient

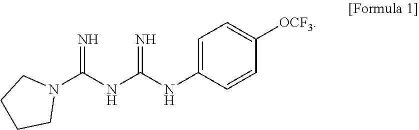 [SMILES: N=C(NC(=N)N1CCCC1)Nc1ccc(OC(F)(F)F)cc1]

19. The method of claim 18, wherein the compound of Formula 1 is administered in combination with encorafenib and binimetinib.

20. The method of claim 18 or 19, wherein the BRAF-mutant melanoma is metastatic.

21. The method of claim 20, wherein the metastasis is to the central nervous system.

22. The method of claim 20 or 21, wherein the metastasis is to the brain.

23. The method of claim 22, wherein the method comprises use of radiation and/or administration of temozolomide.

24. A method of treating castrate-resistant prostate cancer (CRPC), the method comprising administering a pharmaceutical composition to a subject in need thereof with a therapeutically effective dose, wherein the composition comprises a compound of Formula 1 below or a pharmaceutically acceptable salt thereof as an active ingredient

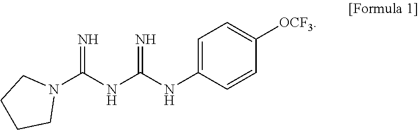 [SMILES: N=C(NC(=N)N1CCCC1)Nc1ccc(OC(F)(F)F)cc1]

25. The method of claim 24, wherein the subject did not respond to at least one line of immunotherapy.

26. The method of claim 24 or 25, wherein the compound of Formula 1 is administered in combination with a checkpoint inhibitor.

27. The method of claim 26, wherein the checkpoint inhibitor is a PD-1 inhibitor.

28. The method of any one of claims 16-27, wherein the compound of Formula 1 is formulated as a tablet or a capsule.

29. The method of claim 28, wherein the tablet or capsule is formulated for delayed release.

30. The method of claim 28 or 29, wherein the tablet or capsule comprises an enteric coating.

31. The method of any one of claims 16-30, wherein the compound of Formula 1 is administered at a dose of about 100 mg, 200 mg, 400 mg, 800 mg, or 1200 mg per day.

32. The method of claim 31, wherein the compound of Formula 1 is administered at a dose of about 800 mg per day.

33. The method of any one of claims 9-32, wherein the pharmaceutically acceptable salt is a salt of an acid selected from the group consisting of formic acid, acetic acid, propionic acid, lactic acid, butyric acid, isobutyric acid, trifluoroacetic acid, malic acid, maleic acid, malonic acid, fumaric acid, succinic acid, succinic acid monoamide, glutamic acid, tartaric acid, oxalic acid, citric acid, glycolic acid, glucuronic acid, ascorbic acid, benzoic acid, phthalic acid, salicylic acid, anthranilic acid, benzensulfonic acid, p-toluenesulfonic acid, methanesulfonic acid, dichloroacetic acid, aminooxy acetic acid, hydrochloric acid, hydrobromic acid, sulfuric acid, phosphoric acid, nitric acid, carbonic acid, and boric acid.

34. The method of claim 33, wherein the pharmaceutically acceptable salt is a salt of acetic acid.

35. The method of any one of claims 1-34, wherein the subject is a human.
